(19) United States
(12) Patent Application Publication
(10) Pub. No.: US 2026/0316062 A1
(43) Pub. Date: Oct. 15, 2026

(54) Mechanically stable VPO catalyst and process for the production thereof

(71) Applicant: Clariant International Ltd, Muttenz (CH)

(72) Inventors: Sebastian BÖCKLEIN, Bruckmühl (DE); Gerhard MESTL, Bruckmühl (DE); Bernadette HOFMANN, Bruckmühl (DE); Gabriele BINDSEIL, Bruckmühl (DE); Anna ADLER, Bruckmühl (DE); Nina RAMMEL, Bruckmühl (DE)

(73) Assignee: Clariant International Ltd, Muttenz (CH)

(21) Appl. No.: 19/117,187

(22) PCT Filed: Oct. 19, 2023
(86) PCT No.: PCT/EP2023/079179

§ 371 Date: Mar. 31, 2025

(30) Foreign Application Priority Data
Oct. 21, 2022 (CN) 202211292982.7
Oct. 21, 2022 (DE) 102022127816.6

(51) Int. Cl. B01J 27/20; B01J 35/30; B01J 35/40; B01J 35/55; B01J 37/00; B01J 37/04; B01J 37/08; B01J 37/16; C07D 307/60

(52) U.S. Cl. CPC B01J 27/20; B01J 35/394; B01J 35/40; B01J 35/55; B01J 37/0009; B01J 37/009; B01J 37/04; B01J 37/08; B01J 37/16; C07D 307/60

(57) ABSTRACT

The invention relates to a VPO catalyst in the form of a shaped body for the oxidation of hydrocarbons with molecular oxygen, especially for the oxidation of butane to maleic anhydride with molecular oxygen, characterized in that the VPO catalyst contains ZnO. The invention further relates to a process for producing a VPO catalyst of the invention in the form of a shaped body, that includes providing an intermediate product comprising vanadyl hydrogen phosphate via steps a)-c), d) drying and calcining the intermediate product at a temperature of max. 300° C. so as to obtain a dried intermediate product, e) shaping the intermediate product from step d) into particles, and f) activating the particles at a temperature above 200° C., in a gas mixture consisting of nitrogen, hydrogen and water vapour, characterized in that ZnO is added after step c), but before step f).

The invention relates to a VPO catalyst in the form of a shaped body for the oxidation of hydrocarbons with molecular oxygen, especially for the oxidation of butane to maleic anhydride with molecular oxygen, characterized in that the VPO catalyst contains ZnO. A VPO catalyst of this kind has enhanced mechanical stability.

The invention further relates to a process for producing a VPO catalyst of the invention, comprising the steps of:
a) providing a reaction mixture that comprises a V(V) compound, a P(V) compound, optionally a Mo compound, a reductant and a solvent,
b) reducing the V(V) compound with the reductant at least in part to vanadyl hydrogen phosphate so as to obtain a suspension of an intermediate product,
c) filtering the suspension of an intermediate product from step b) so as to obtain an intermediate product,
d) drying and/or calcining the intermediate product at a temperature of max. 300° C. so as to obtain a dried intermediate product,
d₁) optionally mixing the dried intermediate product with graphite and/or d₂) compacting and/or granulating the dried intermediate product,
e) shaping the dried intermediate product from steps d) or d₁) and/or d₂) into particles,
f) activating the particles at a temperature above 200° C., in a gas mixture consisting of nitrogen, hydrogen and water vapour,
characterized in that ZnO is added after step c), but before step f).

The invention relates also to the use of ZnO for stabilizing a VPO catalyst in the form of a shaped body and to the use of ZnO as binder for a VPO catalyst in the form of a shaped body.

Maleic anhydride is a chemical intermediate of great economic importance. It is used for example in the production of alkyd and polyester resins, alone or else in combination with other acids. In addition, it is also a versatile intermediate for chemical synthesis, for example for the synthesis of γ-butyrolactone, tetrahydrofuran and butane-1,4-diol, which in turn are used as solvents or may be further processed to afford polymers, for example polytetrahydrofuran or polyvinylpyrrolidone.

Maleic anhydride is generally produced by partial oxidation of n-butane in the gas phase with molecular oxygen or with a gas containing molecular oxygen in the presence of a vanadium-phosphorus oxide catalyst (VPO catalyst) that contains vanadyl pyrophosphate (VPP). Vanadyl pyrophosphate in its pure form contains vanadium with a valency of +4 and is particularly suitable for the production of maleic anhydride from unbranched saturated or unsaturated hydrocarbons having at least four carbon atoms. Fixed-bed reactors and fluidized-bed reactors can both be used.

VPO catalysts have only low intrinsic activity in the reaction of n-butane to maleic anhydride. An adequate conversion therefore requires a large amount of catalyst. Moreover, VPO catalysts rank among the most costly non-noble metal catalysts of all, largely on account of the high cost of their starting materials. This sets the object of improving the catalyst performance (activity and selectivity) and also the lifetime and mechanical stability of such catalysts. It is known from the prior art that the performance of VPO catalysts can be improved by adding foreign elements to the vanadium-phosphorus oxide (VPO) phase, for example through the addition of molybdenum (Mo promoter or Mo doping).

U.S. Pat. No. 5,929,256 discloses the synthesis of an active vanadium-phosphorus catalyst modified with molybdenum for the production of maleic anhydride. In this synthesis, a compound substantially composed of pentavalent vanadium is reacted with a compound containing pentavalent phosphorus in an alcoholic medium suitable for the reduction of vanadium to an oxidation state less than 5. This results in the incorporation of molybdenum into the reaction product, with the formation of a solid precursor composition modified with molybdenum. The alcohol is removed, affording a dried, solid precursor composition modified with molybdenum. This is shaped into shaped bodies comprising the dried, solid precursor composition modified with molybdenum. The dried and shaped precursor compositions modified with molybdenum undergo activation to convert them into the active catalyst.

U.S. Pat. No. 5,070,060 discloses an improvement of the oxidation catalyst that is used for the partial oxidation of n-butane and that comprises mixed oxides of vanadium and phosphorus, zinc and lithium, comprising the addition of an agent for modifying the molybdenum compound in an amount of about 0.005 to 0.025/1 Mo/V to the catalyst during the reaction of the reduced vanadium compound with concentrated phosphoric acid. The addition of Mo generates a catalyst that is very stable, constitutes a more active system and is longer lasting than the unmodified catalyst.

U.S. Pat. No. 3,980,585 discloses a catalyst complex suitable for the conversion of normal C₄ hydrocarbons into maleic anhydride in the gas phase, comprising the components vanadium, phosphorus and copper and also one of the elements selected from Te, Zr, Ni, Ce, W, Pd, Ag, Mn, Cr, Zn, Mo, Re, Sm, La, Hf, Ta, Th, Co, U and Sn, preferably with an alkali metal or an alkaline earth metal.

U.S. Pat. No. 4,056,487 discloses a catalyst that is suitable for the partial oxidation of alkanes to the corresponding anhydrides, for example of normal C₄ hydrocarbons that are to be converted into maleic anhydride in the gas phase, comprising the components vanadium, phosphorus and oxygen, Nb, Cu, Mo, Ni, Co and Cr. Preference is given to components that additionally comprise one or more elements selected from Ce, Nd, Ba, Hf, U, Ru, Re, Li or Mg.

U.S. Pat. No. 4,515,904 discloses a process for producing a phosphorus-vanadium catalyst and a phosphorus-vanadium co-metal catalyst for use in the production of maleic anhydride from butane, the process comprising the reaction of a vanadium compound in an organic ether solvent having about 2 to about 10 carbon atoms with a phosphorus halide at a temperature of about 0° C. to about 200° C. in the presence of water or an aliphatic alcohol having about 1 to about 8 carbon atoms; removal of the solvent; and activation of the catalyst by addition of butane or another hydrocarbon starting product and of a phosphorus compound at an approximate temperature of about 300° C. to about 500° C.

U.S. Pat. No. 5,158,923 discloses an improvement of the oxidation catalyst that is used for the partial oxidation of n-butane and that comprises mixed oxides of vanadium and phosphorus, zinc and lithium, comprising the addition of an agent for modifying the molybdenum compound in an amount of about 0.005 to 0.025/1 Mo/V to the catalyst during the breakdown of the reduced vanadium compound by concentrated phosphoric acid. The addition of Mo generates a catalyst that is very stable, constitutes a more active system and is longer lasting than the unmodified catalyst.

U.S. Pat. No. 5,262,548 discloses an improvement of the oxidation catalyst that is used for the partial oxidation of n-butane and that comprises mixed oxides of vanadium and phosphorus, zinc and lithium, comprising the addition of an agent for modifying the molybdenum compound in an amount of about 0.005 to 0.025/1 Mo/V to the catalyst during the breakdown of the reduced vanadium compound by concentrated phosphoric acid. The addition of Mo generates a catalyst that constitutes a very stable, active system and has a longer lifetime than the unmodified catalyst.

WO 2013062919 A1 discloses a process for producing a promoted VPO catalyst, wherein the catalyst comprises the mixed oxides of vanadium and phosphorus and wherein the catalyst is promoted with at least one selected from niobium, cobalt, iron, zinc, molybdenum or titanium, the process comprising the steps of: (i) preparing a VPO catalyst comprising vanadyl pyrophosphate as principal component and comprising less than 5% by weight of vanadyl phosphate, (ii) contacting the VPO catalyst with a solution comprising as metal source a compound containing at least one metal selected from the group consisting of niobium, cobalt, iron, zinc, molybdenum or titanium so as form a metal-impregnated VPO catalyst, and (iii) drying the metal-impregnated VPO catalyst so as to form the promoted VPO catalyst. In one embodiment, a niobium-activated VPO catalyst is produced.

U.S. Pat. No. 5,280,003 discloses an improvement of an oxidation catalyst that is used for the partial oxidation of n-butane and that comprises mixed oxides of vanadium and phosphorus, zinc, lithium and molybdenum, in which production of the catalyst involves the performance of a crystallization step under static conditions that permit more uniform conditions for crystal growth. The static conditions are maintained by heating the solvent under reflux during crystallization.

U.S. Pat. No. 4,251,390 discloses the improvement of an oxidation catalyst used for the partial oxidation of n-butane that comprises vanadium and phosphorus mixed oxides, characterized in that, during the reaction of the reduced vanadium component with concentrated phosphoric acid, a zinc compound is added to the catalyst in an amount of 0.15 to 0.001 Zn/V. The addition of zinc generates a catalyst that is more readily activated and that is very stable to heating of the reaction system. Small amounts of lithium compounds and silicon compounds also have additional desirable catalytic effects without diminishing the benefit of the zinc compound.

DE 10 2014 004786 A1 relates to a catalyst comprising a vanadium-phosphorus oxide and an alkali metal in which the proportion by weight of the alkali metal in the vanadium-phosphorus oxide is within a range from 10 to 400 ppm based on the total weight of the vanadium-phosphorus oxide, to a process for the production thereof and to the use of the catalyst for the gas-phase oxidation of hydrocarbons, especially for the production of maleic anhydride.

CN 101036891 A discloses a process for the regeneration of a fluidized-bed catalyst for the oxidation of n-butane to maleic anhydride, comprising the following steps: preparing the precursor matrix powder and the auxiliary, mixing the catalyst dusts collected by the fluidized-bed apparatus with the prepared precursor matrix powder and the auxiliary, adding a water-soluble resin glue and stirring under the conditions of an aqueous thermostating, finally shaping by spray-drying so as to obtain the regenerated catalyst. The regenerated catalyst of this invention can be used alongside or in place of the original catalyst in the fluidized-bed reactor and is equivalent therewith in its activity, particle size and use, etc.

CN 1282631 A discloses a vanadium-phosphorus oxide catalyst that is characterized by the addition of zirconium, molybdenum and zinc and in which the vanadium: phosphorus:zirconium:molybdenum:zinc atomic ratio is 1.0:1.0-1.5:0.02-0.06:0.02-0.06:0.02-0.06. Also disclosed is a process for producing the vanadium-phosphorus oxide catalyst, characterized in that 6 parts by weight of vanadium pentoxide and 50-60 parts by weight of concentrated hydrochloric acid are heated under reflux for 1-5 hours and then phosphoric acid is added. The amount added is such that the vanadium to phosphorus atomic ratio is between 1.0:1.0-1.5 and the reflux step is performed for a further 1-5 hours. After cooling, a mixture of zirconium nitrate, ammonium dimolybdate and zinc acetate and 50-200 parts of water is added. The solution, zirconium nitrate, ammonium dimolybdate and zinc acetate are added so as to achieve a vanadium:zirconium:molybdenum:zinc atomic ratio of 1.0:0.02-0.06:0.02-0.06:0.02-0.06; the mixture was then slowly and carefully evaporated in a water bath for 15-30 hours so as to obtain a viscous colloid and the colloid was dried at 120° C. so as to obtain a dark green solid that was the vanadium-phosphorus oxide catalyst of this invention.

CN 111701608 A discloses a method for producing a hydrotalcite-modified vanadium-phosphorus-oxygen catalyst comprising the following steps: preparing a vanadium-phosphorus oxide precursor by mixing a vanadium source, benzyl alcohol and a monohydric C3 to C8 alcohol so as to obtain a mixture, then adding a phosphorus source, heating to 100° C. to 200° C., continuing the reaction, filtering the product and drying so as to obtain the vanadium-phosphorus-oxygen precursor; preparing a hydrotalcite additive by dissolving a water-soluble inorganic zinc salt, an inorganic magnesium salt and an inorganic aluminium salt in water, adding an alkali source, heating to 65° C. to 200° C. so as to permit a reaction, cooling and ageing for 6 to 12 hours, filtering, washing, drying and calcining at a temperature of between 350° C. to 550° C. so as to obtain the hydrotalcite additive, wherein the total amount of zinc and magnesium in the inorganic zinc source, the inorganic magnesium source and the inorganic aluminium source to the amount of aluminium is between 1 to 4:1, the molar ratio between magnesium and zinc is between 1 and 5:10; mixing of the hydrotalcite additive and of the vanadium-phosphorus mixed oxide-oxygen precursor in a mass ratio of 1-10:100, heating to 300-500° C. and calcining so as to obtain a hydrotalcite-modified vanadium-phosphorus-oxygen catalyst.

CN 106938197 A discloses a process for producing a vanadium-phosphorus oxide catalyst. The process comprises the following steps: preparing an oxovanadium hydrogen phosphate hemihydrate catalyst precursor powder doped with a metal auxiliary, wherein the metal auxiliary is at least one selected from Fe, Mo, Co, Ce, Zr, Nb and Ni, and wherein the molar ratio of metal auxiliary to vanadium is between 0.06 to 0.15; and mixing the catalyst precursor powder with a binder and an auxiliary and performing a strand extrusion or a form of tabletting so as to obtain a vanadium-phosphorus oxide catalyst, wherein the binder is at least one selected from phosphoric acid, pyrophosphoric acid, trimethyl phosphate, triethyl phosphate, aluminium dihydrogen phosphate, potassium dihydrogen phosphate, dipotassium hydrogen phosphate, zinc phosphate, tricalcium phosphate, ammonium phosphate, ammonium pyrophosphate and ammonium hexametaphosphate, the auxiliary is at least one selected from graphite, carbon nanotubes, graphene and carbon powder, and the mass ratio between catalyst precursor powder, binder and auxiliary is between 100:(0.1-15):(0.1-10). The process for producing the vanadium-phosphorus oxide catalyst is simple and places no particular demands on the production equipment, and the catalyst obtained has the advantages of high strength, good catalytic activity and facilitation of industrial production and use.

In order to produce VPO catalysts that contain a VPP phase, it is usual to carry out a reduction of vanadium pentoxide (V₂O₅) in the simultaneous presence of phosphoric acid in an organic alcoholic solvent with benzyl alcohol as reductant, resulting in the formation alongside benzaldehyde of vanadyl hydrogen phosphate (VHP). The redox reaction (“reduction”) that proceeds here, in which vanadium in oxidation state V (V(V)) reacts to form the VHP phase, in which vanadyl species (VO²⁺) with vanadium in oxidation state IV (V(IV)) are present, is as follows:

$${V}_{2} {O}_{5} + 2 {H}_{3} P {O}_{4} + Ph - C {H}_{{2}^{-}} O H \to 2 {VOHPO}_{4}^{⋆} {½ H}_{2} O + Ph - CHO + 2 {H}_{2} O \qquad ( 1 )$$

In a subsequent activation step, the VHP phase is converted by the action of heat into the vanadyl pyrophosphate phase alongside the elimination of water.

$$2 {VOHPO}_{4}^{⋆} {½ H}_{2} O \to {( VO )}_{2} {P}_{2} {O}_{7} + 1 {½H}_{2} O \qquad ( 2 )$$

A problem in the catalytic reaction of butane to maleic anhydride using VPO catalysts is that the process must be operated in a regime that is limited by pore diffusion. The porosity thus has a direct influence on the catalytic yield here. Care must therefore be taken to ensure that the pore structure is not adversely influenced during shaping (for example by tabletting). This does however have the consequence that the mechanical stability of the shaped bodies suffers. This must nevertheless be high enough for the tablets to survive the filling process (drop into a reaction tube approx. 6 m in length) intact. Otherwise, the smaller fragments would result in the dynamic pressure in the process being too high, leading to high compressor costs or reduced throughputs. A technical object is thus to increase the mechanical strength of the shaped bodies without adversely affecting the pore structure.

The object of the present invention was therefore to provide an improved VPO catalyst for the gas-phase oxidation of hydrocarbons, especially for the production of maleic anhydride, that has comparable porosity and catalytic performance compared to catalysts commonly used up to now and at the same time has substantially improved mechanical strength.

The object is achieved by a VPO catalyst in the form of a shaped body for the oxidation of hydrocarbons with molecular oxygen, especially for the oxidation of butane to maleic anhydride with molecular oxygen, characterized in that the VPO catalyst contains ZnO.

The VPO catalyst according to the invention includes the VPO phase or comprises or consists essentially thereof. For example, the VPO catalyst according to the invention includes the VPO phase in a content of more than 70% by weight, preferably more than 80% by weight, particularly preferably more than 90% by weight, based on the total weight of the VPO catalyst. In addition, the VPO catalyst may include VPP, dopings and unreacted oxides of the starting materials, for example vanadium pentoxide or phosphorus oxide.

The VPO catalyst according to the invention may optionally contain between 0.1% by weight and 1% by weight, preferably between 0.4% by weight and 0.7% by weight, of Mo, based on the total weight of the VPO catalyst.

The VPO catalyst according to the invention may however also contain alkali metals such as Na and/or K, preferably 80 to 300 ppm of alkali metal. In addition, the VPO catalyst according to the invention may also contain carbon, for example in the form of graphite, for example in an amount of from 3% by weight to 5% by weight based on the total weight of the catalyst. The graphite present serves for example as a tabletting aid.

The VPO catalyst according to the invention includes ZnO (zinc (II) oxide), but it is possible for further zinc compounds to be additionally present. The content of Zn in the VPO catalyst arising through the presence of ZnO and possibly further Zn compounds must be between 0.2% by weight and 10% by weight, preferably between 0.5% by weight and 7% by weight, of Zn, more preferably between 1% by weight and 5% by weight, in each case based on the total weight of the catalyst.

The VPO catalyst preferably has the following elemental composition:
0.5% by weight to 7% by weight of Zn,
0% by weight to 0.7% by weight of Mo,
26% by weight to 31% by weight of V,
17% by weight to 21% by weight of P,
3% by weight to 5% by weight of C,
the rest being oxygen, in each case based on the total weight of the VPO catalyst.

The VPO catalyst according to the invention comprises Zn primarily in the form of ZnO and—provided the content of the ZnO phase is sufficiently high—the catalyst exhibits in an XRD powder diffractogram recorded using Cu-Kα radiation reflections typical of a ZnO phase. In particular, sharp reflections at 31.7° to 31.9°, 34.3° to 34.5°, 36.2° to 36.4° are detected.

The ZnO has the effect according to the invention of stabilizing the catalyst particles so that they have a higher mechanical strength compared to the strength when these compounds are absent. Preferably, the ZnO serves to stabilize a VPO catalyst in particle form.

The VPO catalyst according to the invention is present in the form of shaped bodies; the shape of the shaped bodies can be of varying design, depending on the desired contact time, flow rate and dynamic pressure during the catalytic reaction. The VPO catalyst in the form of a shaped body is understood as meaning shaped bodies produced by a shaping step such as tabletting. When present in large quantity in a tube-bundle reactor, the shaped bodies according to the invention form a layer or a catalyst bed through which the reactants butane, especially n-butane, and air are passed.

For example, the shaped bodies according to the invention may be in the form of a customary cylindrical shape. The cylinder in that case has a height (length along the cylinder axis) of 3 mm to 8 mm and an essentially round base surface with a diameter of 3 mm to 8 mm. Preferably, the cylinder has a central axial opening; this can have for example a diameter of 1 mm to 3 mm.

For example, the shaped body according to the invention may have a height of 4.7 mm, an external diameter of 4.7 mm and a central axial opening with a diameter of 1.3 mm. This shaped body then has a geometric surface area of 1.2 cm², a volume of 0.075 cm³ and a mass of 0.12 g. Filling a large quantity of such shaped bodies into a 21 mm reactor results in a poured density of about 0.85 g/cm³ to 0.89 g/cm³.

For example, the shaped bodies according to the invention may have a height of 5.6 mm, an external diameter of 5.5 mm and a central axial opening with a diameter of 2.3 mm. These shaped bodies then have a geometric surface area of 1.77 cm², a volume of 0.111 cm³ and a mass of 0.18 g. Filling a large quantity of such shaped bodies into a 21 mm reactor results in a poured density of about 0.72 g/cm³ to 0.76 g/cm³.

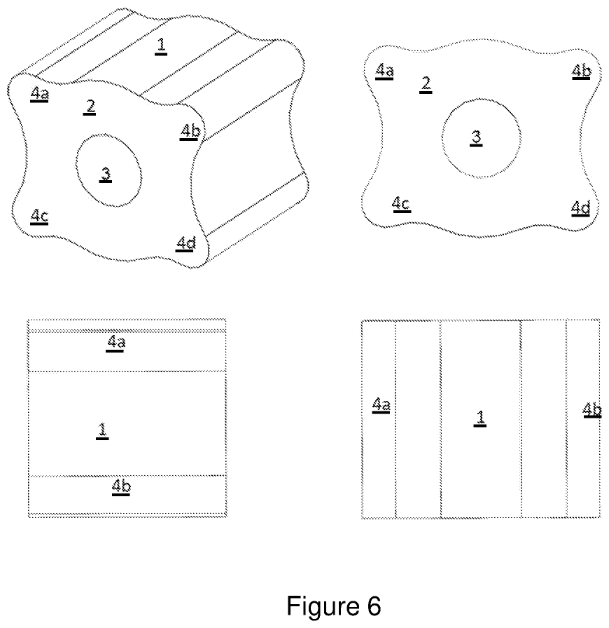

Preferred shaped bodies for use in the reactor concept according to the invention are those described in EP 2643086 A1. The preferred double-alpha shape is especially characterized in that each individual shaped body is in each case designed in the form of a cylinder having an outer base surface [1], a cylinder surface [2], a cylinder axis and at least one continuous opening [3] running parallel to the cylinder axis, and the outer base surface [1] of the cylinder contains at least four lobes [4a, 4b, 4c, 4d], wherein a geometric base body enclosing the shaped bodies is a prism having a prism base surface having a length and a width, wherein the length is greater than the width and wherein the lobes [4a, 4b, 4c, 4d] are enclosed by prism corners of the prism base surface (FIG. 6).

Preference is given to a shaped body in a double-alpha shape with a height (length along the cylinder axis) of 3 mm to 8 mm, a length of 5 mm to 9 mm and a width of 4 mm to 8 mm and an internal hole diameter of 1 mm to 4 mm. Preference is for example given to a shaped body in a double-alpha shape with a height of 5.6 mm, a length of 6.7 mm, a width of 5.8 mm and an internal hole diameter of 2.1 mm. These shaped catalyst bodies have a geometric surface area of 2.37 cm², a volume of 0.154 cm³ and a mass of 0.24 g. Filling into a 21 mm reactor results in a poured density of about 0.60 g/cm³ to 0.62 g/cm³.

The VPO catalyst/shaped body of the invention has a side crush strength of more than 25 N, preferably between 25 N to 200 N. Particularly preferably, the VPO catalyst/shaped body has a side crush strength of more than 30 N to 150 N, even more preferably of more than 35 N to 100 N.

The VPO catalyst/shaped body of the invention in a cylinder shape has a side crush strength of more than 25 N, preferably between 25 N to 50 N. Particularly preferably, the VPO catalyst/shaped body has a side crush strength of more than 30 N to 45 N, even more preferably of more than 35 N to 40 N.

The VPO catalyst/shaped body of the invention in a double-alpha shape has a side crush strength of more than 50 N, preferably between 50 N to 200 N. Particularly preferably, the VPO catalyst/shaped body has a side crush strength of more than 100 N to 170 N, even more preferably of more than 120 N to 150 N.

The invention further relates to a process for producing a VPO catalyst of the invention in the form of a shaped body, comprising the steps of:
a) providing a reaction mixture that comprises a V(V) compound, a P(V) compound, optionally a Mo compound, a reductant and a solvent,
b) reducing the V(V) compound with the reductant at least in part to vanadyl hydrogen phosphate so as to obtain a suspension of an intermediate product,
c) filtering the suspension of an intermediate product from step b) so as to obtain an intermediate product,
d) drying and/or calcining the intermediate product at a temperature of max. 300° C. so as to obtain a dried intermediate product,
d1) optionally mixing the dried intermediate product with graphite and/or
d2) compacting or granulating the dried intermediate product,
e) shaping the dried intermediate product from steps d) or d1) and/or d2) into a shaped body,
f) activating the shaped body at a temperature above 200° C., in a gas mixture consisting of nitrogen, hydrogen and water vapour,
   characterized in that ZnO is added after step c), but before step f).

In a process step a), the starting materials for the reduction step b) are provided in the reaction mixture so as to carry out the reduction. According to the invention, the reaction mixture comprises as starting materials a V(V) compound, a P(V) compound, optionally a Mo compound, a reductant and a solvent, preferably the reaction mixture consists of these starting materials. For example, the reaction mixture may consist of 45% by weight to 90% by weight of solvent, 5% by weight to 15% by weight of reductant, 5% by weight to 15% by weight of a V(V) compound, up to 1% by weight of a Mo compound and between 5% by weight to 25% by weight of a P(V) compound. More specifically, 60% by weight to 70% by weight of isobutanol, 5% by weight to 15% by weight of benzyl alcohol, 5% by weight to 15% by weight of vanadium pentoxide, 0.05% by weight to 0.2% by weight of (NH₄)₂MoO₄ and 10% by weight to 20% by weight of phosphoric acid, in each case based on the total weight of the reaction mixture, may for example be initially charged as the reaction mixture.

The V(V) compound used as the starting material in the reaction mixture according to step a) is a compound containing vanadium in oxidation state V and is preferably V₂O₅.

The P(V) compound used as the starting material in the reaction mixture according to step a) is a compound containing phosphorus in oxidation state V and is preferably phosphoric acid or a phosphate salt such as Na₃PO₄. The phosphoric acid (H₃PO₄) used is preferably anhydrous (100% phosphoric acid) or phosphoric acid containing only small amounts of water, i.e. phosphoric acid having a concentration of 98 to 100%, preferably 99 to 100% (percent values refer to the percent content by weight of pure phosphoric acid in relation to the weight of the water-phosphoric acid mixture, as customarily expressed). Alternatively, for the preparation of the reaction mixture according to step a) it is possible to use phosphoric acid having a concentration of more than 100%, which reacts immediately with any water initially present in the reaction mixture according to step a), with the formation of phosphoric acid having a concentration of 98 to 100%, preferably 99 to 100%, more preferably 100%, with the result that the reaction mixture according to step a) preferably does not contain phosphoric acid having a concentration of more than 100% and at the same time not more than 0.2% by weight of water, based on the weight of the reaction mixture, remains in the reaction mixture.

The molybdenum compound that may optionally be used as starting material in the reaction mixture according to step a) is any desired compound that contains molybdenum, for example molybdenum trioxide, ammonium heptamolybdate ((NH₄)₆Mo₇O₂₄)*4H₂O), ammonium paramolybdate ((NH₄)₆Mo₇O₂*4H₂O), meta molybdate, molybdic acid (H₂MoO₄) and salts thereof, such as (NH₄)₂MoO₄, Na₂MoO₄, K₂MoO₄ or (NH₄)₂Mo₂O₇.

The reductant present in the reaction mixture according to step a) may be any desired reductant capable of reducing the V(V) compound such that vanadyl hydrogen phosphate forms at least in part. Preferably, the reductant is an organic reductant such as ethanol, isobutanol or an aromatic alcohol, including in particular benzyl alcohol.

The solvent present in the reaction mixture according to step a) is preferably an alcohol, more preferably a high-boiling aliphatic alcohol, especially isobutanol, alternatively ethanol or isopropanol.

The starting materials are provided in an appropriate reaction vessel suitable for the performance of the subsequent step b), i.e. the heating of the reaction mixture to above room temperature, for example up to a temperature of 100° C. Since the reduction is preferably carried out with stirring in a reflux step, the reaction vessel preferably has a reflux condenser and a device for stirring the reaction mixture. Optionally, the reaction vessel is equipped with a device that permits the removal from the reaction mixture of the water formed during the reduction, i.e. a water separator, for example a Dean-Stark trap.

Effected in process step b) is the at least partial reduction of the V(V) compound to an intermediate product that comprises a vanadyl hydrogen phosphate phase and optionally molybdenum and that, together with the solvent and the other partially reacted components from step a), forms the suspension of the intermediate product. This reduction is effected preferably under reflux at standard pressure, the temperature being increased here in line with the boiling point of the solvent used; it is preferable that the process according to the invention involves the performance of just a single reflux step. The intermediate product preferably contains vanadyl hydrogen phosphate as the main phase or can even consist essentially of a vanadyl hydrogen phosphate phase. The molybdenum possibly likewise present in the intermediate product may be present in the form of doping of the vanadyl hydrogen phosphate phase, molybdenum doping being understood as meaning that the molybdenum is either incorporated into the vanadyl hydrogen phosphate phase or is present on the surface thereof. In addition to the vanadyl hydrogen phosphate phase, it is however possible for the reduction to result in the formation of further vanadium-phosphorus mixed oxides in which vanadium has an oxidation state of IV or even III. The reduction does not need to proceed to completion, which means that proportions of the V(V) compound and of the P(V) compound also remain in the intermediate product and thus in the suspension of the intermediate product. In the intermediate product of the reduction, vanadium is however typically present in an average oxidation state of 3.8 to 4.2.

The water formed during the reduction may be removed from the reaction mixture during the reduction. The removal of water during the reduction is in the prior art effected either physically, for example with the aid of a water separator, or chemically through the use of compounds that bind the water, for example drying agents or anhydrides such as phosphoric acid having a concentration of more than 100%. The reaction mixture according to step a) may comprise, for example, anhydrides that react with and bind water, in particular, the reaction mixture according to step a) may comprise phosphoric acid having a concentration of more than 100%.

Process step c), i.e. the filtration of the suspension of the intermediate product, takes place for example in an atmosphere of an inert gas such as nitrogen or a noble gas. In this context, inert gas means any gas that does not react with the intermediate product under the specified conditions during the filtration, but at the same time displaces oxygen from the air so as to minimize the risk of an explosion. The filtration is carried out by means known to those skilled in the art, typically by means of a filter press, a decanter or through a filter funnel. Process step c) affords the uncalcined intermediate product, which is still wet with solvent.

The drying of the intermediate product obtained by the filtration (the solid filtration residue) in process step d) is effected at a temperature above room temperature, typically at a temperature of up to 150° C. under reduced pressure/vacuum or under inert gas, so as to obtain a dried intermediate product (dried intermediate product from step d)). In this context, inert gas means any gas that does not react with the intermediate product under the conditions of the drying, but at the same time displaces oxygen from the air so as to minimize the risk of an explosion, for example nitrogen or a noble gas. Preferably drying proceeds under reduced pressure/vacuum between 50° C. and 150° C., preferably between 90° C. and 140° C.

Alternatively or optionally, drying can according to step d) be followed by calcining. The calcining is effected at elevated temperature between 150° C. and 350° C., preferably between 230° C. and 290° C., under inert gas. In this context, inert gas means any gas that does not react with the intermediate product under the conditions of the calcining, but at the same time displaces oxygen from the air so as to minimize the risk of an explosion, for example nitrogen or a noble gas.

Optionally, process step d) may be followed by one or more of process steps d₁) and/or d₂):According to optional process step d₁), 1% by weight to 10% by weight of graphite is added to the dried intermediate product so as to obtain an intermediate product mixture (dried intermediate product from step d₁)). According to optional process step d₂), the dried intermediate product from step d) or d₁) undergoes a compaction and/or granulation so as to obtain a compacted or granulated dried intermediate product (dried intermediate product from step d₂)). The dried intermediate product from step d) or d1) is for example compacted into plates with a roller compactor at a compaction pressure of 190 bar, a gap width of 0.60 mm and a roller speed of 7 rpm and granulated through a 1 mm screen.

In process step e), the dried intermediate product from step d), d₁) or d₂) is shaped into shaped catalyst bodies; this can be effected for example through tabletting. This is accomplished for example by pressing the granulate with a rotary tablet press into the desired tablet shape having the appropriate height.

In the subsequent process step f), the shaped catalyst bodies obtained undergo activation at a temperature above 200° C. The activation is effected typically in a gas mixture consisting of air, inert gas and water vapour, wherein the inert gas used may be any gas that does not react with the shaped catalyst body under the specified conditions during the activation and is particularly preferably nitrogen or a noble gas. Alternatively, the activation may also be effected in process gas, i.e. in a gas mixture containing air and butane. The activation is effected at a temperature within a range from 300° C. to 500° C., preferably within a range from 350° C. to 450° C. Activation affords the finished VPO catalyst as the product of the process. When it is the graphite-containing intermediate product mixture from step d1) or d2) that has been formed into shaped bodies in process step e) that is activated, a graphite-containing VPO catalyst is obtained as the product of the process. The tableted end product typically has a side crush strength of above 25 N, typically between 25 N to 200 N.

The process according to the invention for producing the VPO catalyst in the form of a shaped body is characterized in that ZnO (zinc (II) oxide) is added after process step c) but before process step f), the ZnO being added preferably in the form of a pulverulent solid. The ZnO may also be added as a component of a binder having further constituents; in a preferred embodiment, the binder comprises in addition to ZnO one or more further metal compounds. Preferably, a solid Mg compound, for example MgO, is present alongside the ZnO present in the binder.

The ZnO, or the binder comprising ZnO, is preferably added in powder form, but it is also possible for the binder comprising ZnO to be present in the form of a liquid, for example as a suspension. In the simplest case, ZnO particles or ZnO crystallites are suspended undissolved in a liquid medium such as water. It is however also possible for the binder comprising ZnO to include organic constituents.

The addition of the ZnO, or of the binder comprising ZnO, is expediently effected by mixing the e.g. pulverulent binder with the intermediate product or with the shaped bodies from the respective process step. This can also be effected with the aid of a metering device and a mixer. In order to produce the catalyst of the invention, sufficient ZnO, or binder comprising ZnO, is generally added. For example, 0.5% by weight to 7% by weight, preferably 1% by weight to 5% by weight, more preferably 2% by weight to 4% by weight, of ZnO, based on the weight of the dried intermediate product from steps d), d1) or d2 (without ZnO), may be added.

The invention relates also to the use of ZnO in solid form for stabilizing a VPO catalyst in the form of a shaped body and to the use of ZnO as binder for a VPO catalyst in the form of a shaped body.

As described in the invention, ZnO may be used as a binder for a VPO catalyst, thereby increasing the mechanical stability of the VPO catalyst in the form of a shaped body. Preference is given to using the ZnO in powder form in a process described according to the invention and adding this between steps c) to f), i.e. after step c) but before the performance of step f) or preferably between steps c) to e) or during one of steps c), d) or e).

The invention further relates to a process for producing maleic anhydride by catalytic oxidation of n-butane, wherein a reactant gas comprising oxygen and n-butane is passed through a reactor tube containing a bed of the VPO catalysts of the invention.

The bed of the VPO catalysts in the reactor tube consists of the VPO catalysts/shaped bodies of the invention, which are poured into the reactor tubes and, after resting, afford a catalyst bed. The reactor tube is preferably part of a large number of reactor tubes of a tube-bundle reactor, such as ones for the industrial production of maleic anhydride known to those skilled in the art. During the reaction, the bed of the VPO catalysts is present at a temperature of between 300° C. and 420° C. The reactant gas may contain for example between 0.2% to 10% by volume of n-butane and between 5% and 50% by volume of oxygen and is passed through the reactor tube at a space velocity of 1100 h⁻¹ to 2500 h⁻¹, preferably 1300 h⁻¹ to 2000 h⁻¹.

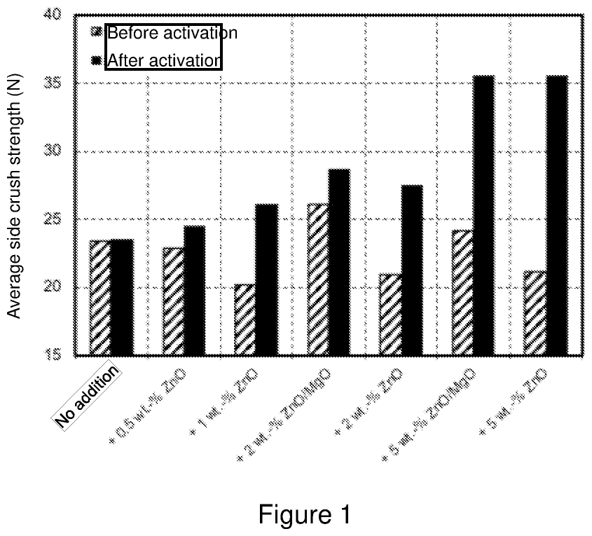

FIG. 1: Influence of the use of ZnO and ZnO/MgO solids in the synthesis on the stability of the VPO catalysts (examples 1 to 7).

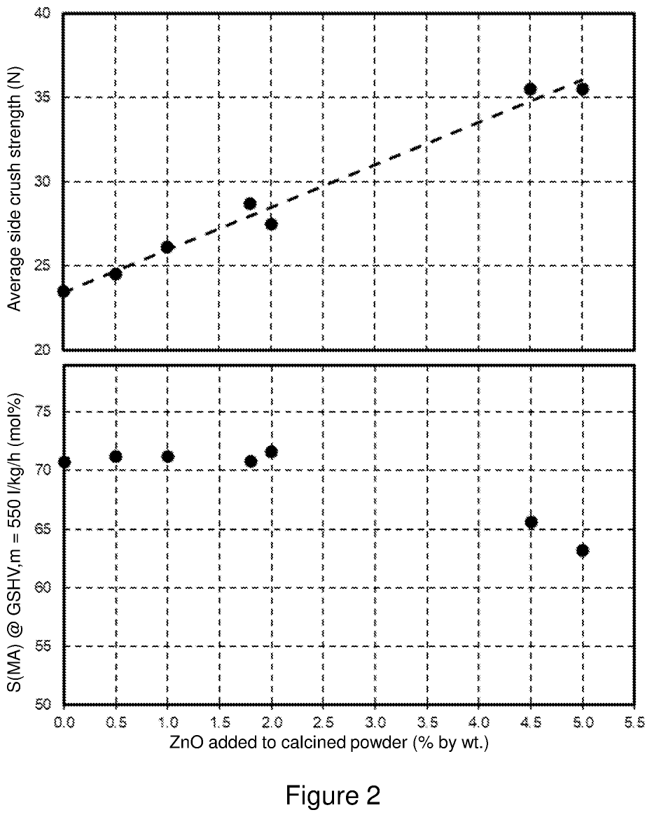

FIG. 2: Change in the side crush strength and selectivity of the VPO catalysts with varying amounts of added Zn.

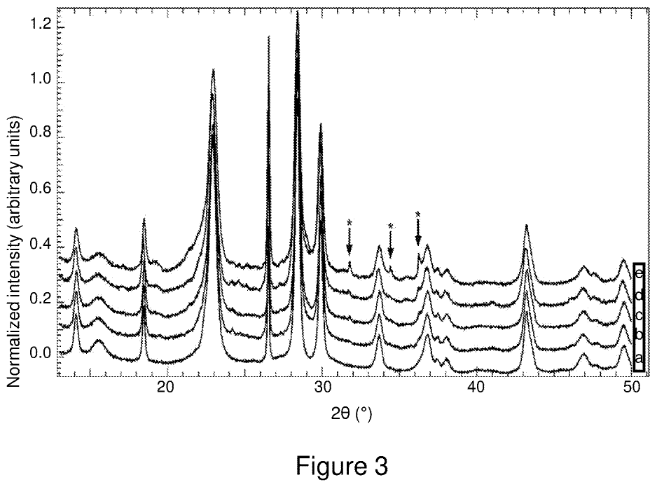

FIG. 3: XRD diffractograms of the VPO catalysts according to examples 1 to 5.

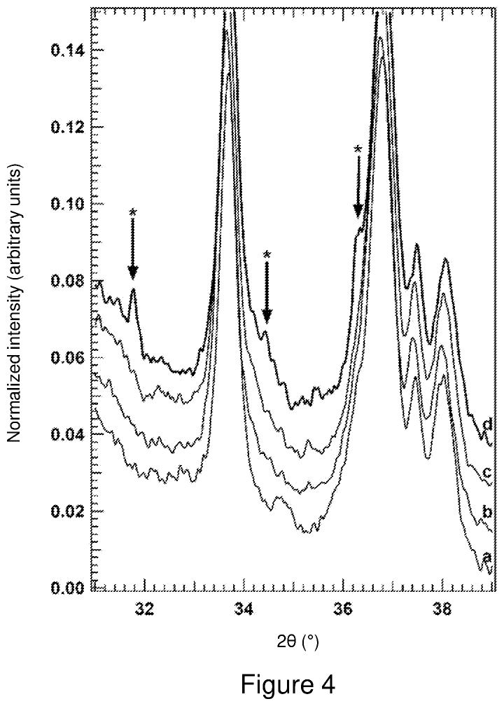

FIG. 4: XRD diffractograms of the VPO catalysts with addition in each case of 2% by weight of ZnO a) at the start of the reflux step, b) after filtration and before vacuum drying, c) after vacuum drying and before calcining, d) after calcining and before tabletting (in each case according to examples 8 to 10 and 5).

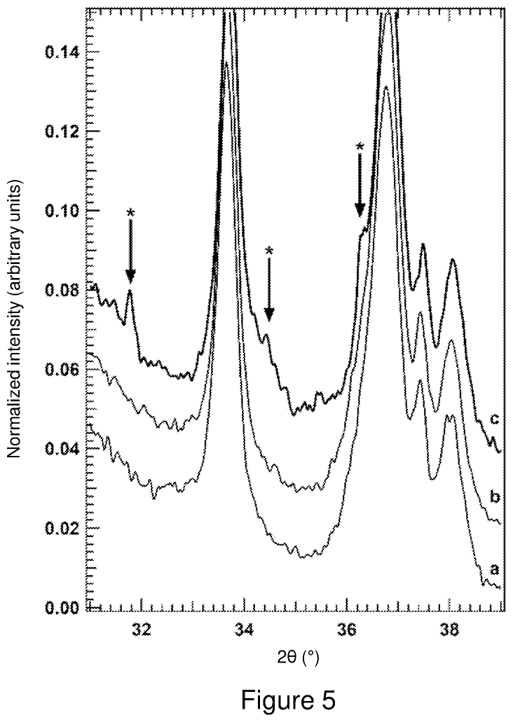

FIG. 5: XRD diffractograms of the VPO catalysts a) according to example 11, b) according to example 12 and c) according to example 5.

FIG. 6: Representations of the preferred catalyst particle, the “double-alpha shape” from four different perspectives.

### EXAMPLES

### Example 1 (Comparative)

### Apparatus Used

A heating mantle, inside which is a 2 L four-necked flask, is positioned on a laboratory jack. Present in the central neck of the four-necked flask is a half-moon impeller with corresponding stirrer seal that is connected to the stirrer unit by means of a stirrer coupling. Present in the right-hand neck is a thermometer and in the left-hand neck a riser to a reflux condenser. The frontally positioned central neck is used for filling with the chemicals, after which the nitrogen inlet is connected there. The entire apparatus can also be flushed with nitrogen. The nitrogen for this is first passed through a gas-wash bottle and then into the apparatus and passes out at the top of the condenser, again through a gas-wash bottle.

Preparation of the reaction mixture and reduction (process steps a) and b).

1069.5 g of isobutanol and 156.0 g of benzyl alcohol are first added. 150 g of V₂O₅ is added with stirring. The V₂O₅ addition is followed by addition of 2.52 g of ammonium dimolybdate. 232.50 g of phosphoric acid (100%, anhydrous) is then added to the suspension and the mixture is heated under reflux under N₂ for 10 h.

### Filtration (Process Step c))

After cooling the suspension of the intermediate product, this is transferred from the four-necked flask to a filter funnel and the liquid is removed by suction. The damp filter cake is pressed dry overnight in a press at 14 to 18 bar.

### Drying/Calcining (Process Step d)):

The pressed filter cake is transferred to the evaporator flask of a rotary evaporator. The filter cake is dried at 110° C. overnight under water-jet vacuum. The powder dried in this way is placed in a furnace in a suitable calcining pot and calcined in an N₂ atmosphere at temperatures of 200° C. to 300° C. for 9 h.

Compacting/Tableting (Process Steps d₁, d₂ and e)):

Before compacting/tableting, 5% by weight of graphite is added to the calcined pulverulent intermediate product and this is mixed homogeneously using a drum hoop mixer. This powder is compacted into plates with a roller compactor at a compaction pressure of 190 bar, a gap width of 0.60 mm and a roller speed of 7 rpm and granulated through a 1 mm screen.

The granulate is pressed with a rotary tablet press into the desired tablet shape having the appropriate height, for example 5.6×5.6×2.3 mm, and side crush strength.

### Activation to Pyrophosphate (Process Step f)):

The activation to form vanadium pyrophosphate is carried out under controlled conditions in a retort installed in a programmable furnace. The calcined tablets are evenly filled into the retort and the retort is sealed tightly. The catalyst is then activated in a moist air/nitrogen mixture (50% atmospheric humidity), initially at over 300° C. for 5 h and then at over 400° C. for 9 h.

### Examples 2 to 7 (Inventive)

The inventive VPO catalysts were produced in analogous manner to example 1, with the exception that the calcined powder from process step d) was mixed per 100 g with 4.5 g (example 2), 1.8 g (example 3), 5.0 g (example 4), 2.0 g (example 5), 1.0 g (example 6), 0.5 g (example 7) of ZnO powder (commercial ZnO, particle size<100 μm, impurity content less than 0.05% by weight) and 0.5 g (example 2) and 0.2 g (example 3) of MgO powder and at the same time with the graphite. This afforded calcined precursor powders containing 5.0% by weight of ZnO/MgO (example 2), 2.0% by weight of ZnO/MgO (example 3), 5.0% by weight of ZnO (example 4), 2.0% by weight of ZnO (example 5), 1.0% by weight of ZnO (example 6) and 0.5% by weight of ZnO (example 7).

The breaking strength of VPO catalysts according to examples 1 to 7 was in each case tested before and after activation (process step f)). XRD diffractograms of the VPO catalysts were generated and these were tested in respect of catalytic selectivity.

The results presented in Table 1 and FIG. 1 show that the addition of ZnO or ZnO/MgO after the calcining step results in no systematic increase in side crush strength (SCS) being observed in a measurement immediately after tableting. Surprisingly, this is however the case after the VPO catalyst tablets had undergone the subsequent activation step. Here, the particles with addition of ZnO or ZnO/MgO show a systematic and very clear increase in side crush strength.

Alongside the increase in mechanical strength, the maintenance of catalytic performance despite this addition is key. The results of the catalytic test reaction in FIG. 2 show that the stabilized shaped catalyst bodies of the invention exhibit usable catalytic performance.

It can be seen from the XRD diffractograms in FIG. 3 that the addition of ZnO results in the following characteristics:
31.7° to 31.9° (sharp reflection, ZnO)
34.3° to 34.5° (sharp reflection, ZnO)
36.2° to 36.4° (sharp reflection, ZnO)

These characteristics become more intense with increasing ZnO content and are in good agreement with ZnO (in wurtzite structure/as zincite).

### Comparative Example 8, Examples 9, 10

To examine the correlation between the presence of the stabilizing ZnO phase and the time of addition of the binder, XRD diffractograms were recorded after different times of addition. Whereas in examples 2 to 7 ZnO was always added after calcining and before tabletting, the ZnO was added at the time of preparation of the reaction mixture during process step a), i.e. together with the addition of the V₂O₅ (comparative example 8), immediately after the filtration according to process step c) (example 9), or during process step d), i.e. after vacuum drying and before calcining (example 10). In these examples, 5.2 g of ZnO was in each case added, which means that the finished catalysts nominally contain in each case 2% by weight of ZnO.

FIG. 4 shows that the XRD reflections observed when ZnO is added after calcining and before tabletting, in particular those at 31.7 to 31.9°, 34.3 to 34.5°, 36.2 to 36.4°, are not visible when the ZnO is added at the start of the reflux step. (FIG. 4, diffractogram a corresponding to comparative example 8).

### Comparative Examples 11 and 12

The VPO catalysts according to comparative examples 11 and 12 were produced in analogous manner to the VPO catalyst according to example 1, but with the activated tablets impregnated with Zn(OAc)₂ solution. Two different concentrations were used here so as to obtain a Zn/V ratio respectively of 0.008 (=0.29% by weight of Zn based on the total catalyst weight) and 0.016 (corresponding to 0.58% by weight based on the total catalyst weight). After impregnation the catalysts were dried in air, first for 18 h at 100° C. and then for a further 20 min at 350° C.

FIG. 5 shows the XRD diffractograms of the VPO catalysts according to comparative examples 11 and 12 in a comparison with the VPO catalyst according to example 5 produced according to the invention. It can be seen that addition of the ZnO compound to the activated tablets does not give rise to any reflections, in particular at 31.7 to 31.9°, 34.3 to 34.5°, 36.2 to 36.4°.

### Comparative Example 13 and 13a

The VPO catalyst according to comparative example 13 was produced in analogous manner to the VPO catalyst according to example 2, but with 10% by weight of binder of the Secar 71 type and 4% by weight of graphite added to the calcined powder.

The stability of the VPO catalyst can be determined very easily by measuring the stability in respect of the by-product water, since a significant proportion of water vapour originating from the oxidation reaction is present during the reaction, which can damage the catalyst. The stability in respect of the by-product water of the inventive VPO catalyst according to example 4 was therefore tested in comparison with a VPO catalyst according to comparative example 13 produced with conventional binder. For this, the selectivity of the untreated samples was tested, then both samples were impregnated with water and the selectivity test repeated (comparative example 13a). It was found that impregnation with water does not result in the catalyst of the invention incurring any loss of selectivity within the tolerance of the measurement.

### Examples 14 and 15

The VPO catalysts according to examples 14 and 15 were produced in analogous manner to the VPO catalyst according to example 1, but without addition of ammonium dimolybdate, with the result that the VPO catalyst obtained was free of Mo.

In addition, 1% by weight (example 14) or 2% by weight (example 15) of ZnO, based on the total weight of the calcined powder, was added to the calcined powder after calcining and before tabletting. As shown by Table 1, an increase in side crush strength was achieved also in the case of the Mo-free VPO catalysts.

### Example 16 (Comparative)

### Apparatus Used

The same apparatus was used as in example 1.

Preparation of the reaction mixture and reduction (process steps a) and b).

First, the four-necked flask is charged with 150 g of V₂O₅. To this is added 300 mL of benzyl alcohol and 1200 mL of isobutanol. The suspension is inertized with N₂ and then heated under reflux for 10 h while stirring. After cooling to max. 40° C. while stirring, 7.51 g of ammonium dimolybdate, 6.60 g of iron (III) nitrate nonahydrate, 2.55 g of cerium (III) nitrate hexahydrate and 4.5 g of niobium ammonium oxalate are added. After adding 138 mL of phosphoric acid (85%) over a 15-minute period while stirring and inertizing with N₂, the suspension is heated under reflux for a further 24 h while stirring.

### Filtration (Process Step c))

After cooling the suspension of the intermediate product, this is transferred from the four-necked flask to a filter funnel and the liquid is removed by suction. The damp filter cake is washed once with ethanol (100%) and the liquid in the filter funnel again removed by suction. The same procedure is then repeated with double-distilled water.

### Drying/Calcining (Process Step d)):

The washed filter cake is transferred to the evaporator flask of a rotary evaporator. The filter cake is dried at 120° C. overnight under water-jet vacuum. The powder dried in this way is placed in a furnace in a suitable calcining pot and calcined in an N₂ atmosphere at temperatures of 200° C. to 300° C. for 9 h.

Compacting/Tableting (Process Steps d₁, d₂ and e)):

Before compacting/tableting, first 2.1% by weight of Zn₃(PO₄)₂ and then 4% by weight of graphite are added to the calcined pulverulent intermediate product and this is mixed homogeneously using a drum hoop mixer. This powder is compacted into plates with a roller compactor at a compaction pressure of 190 bar, a gap width of 0.60 mm and a roller speed of 7 rpm and granulated through a 1 mm screen.

The granulate is pressed with a rotary tablet press into the desired tablet shape having the appropriate height, for example 5.6×5.6×2.3 mm, and side crush strength.

### Heat-Treatment

The following heat-treatment is carried out under controlled conditions in a retort installed in a programmable furnace. The calcined tablets are evenly filled into the retort and the retort is sealed tightly. The catalyst is then treated at 120° C. for 18 h in a stream of air.

### Example 17 (Comparative)

The VPO catalyst according to example 17 was produced in analogous manner to the VPO catalyst according to example 1, but with 2.1% by weight of Zn₃ (PO₄)₂ based on the total weight of the calcined powder added to the calcined powder after calcining and before tabletting.

TABLE 1
Summary of the results

| | | Amount | Side crush | Side crush | | |
|---|---|---|---|---|---|---|
| | | of | strength | strength | | |
| | | binder | before | after | | Selectivity |
| | | [% by | activation | activation | XRD | (MA) |
| Example | Binder | wt.] | [N] | [N] | FIG. | [%] |
| 1 | — | 0.0 | 23.4 | 23.5 | 3 | 70.7 |
| (comparative) | | | | | | |
| 2 | 9ZnO/ | 5.0 | 24.1 | 35.5 | 3 | 65.6 |
| (inventive) | 1MgO* | | | | | |
| 3 | 9ZnO/ | 2.0 | 26.1 | 28.7 | 3 | 70.8 |
| (inventive) | 1MgO* | | | | | |
| 4 | ZnO | 5.0 | 21.1 | 35.5 | 3 | 63.2 |
| (inventive) | | | | | | |
| 5 | ZnO | 2.0 | 20.9 | 27.5 | 3, 4, 5 | 71.6 |
| (inventive) | | | | | | |
| 6 | ZnO | 1.0 | 20.2 | 26.1 | — | 71.2 |
| (inventive) | | | | | | |
| 7 | ZnO | 0.5 | 22.8 | 24.6 | — | 71.2 |
| (inventive) | | | | | | |
| 8 | ZnO | 2.0 | — | — | 4 | — |
| (comparative) | | | | | | |
| 9 | ZnO | 2.0 | — | — | 4 | — |
| (inventive) | | | | | | |
| 10 | ZnO | 2.0 | — | — | 4 | — |
| (inventive) | | | | | | |
| 11 | Zn(OAc)₂ | 0.3 | — | — | 5 | — |
| (comparative) | | | | | | |
| 12 | Zn(OAc)₂ | 0.6 | — | — | 5 | — |
| (comparative) | | | | | | |
| 13 | Secar | 10 | — | — | — | — |
| (comparative) | | | | | | |
| 13a | Secar | 10 | — | — | — | — |
| (comparative) | | | | | | |
| 14 | ZnO | 1.0 | 17.6 | 23.3 | 2 | — |
| (inventive) | | | | | | |
| 15 | ZnO | 2.0 | 15.7 | 27.5 | 2 | — |
| (inventive) | | | | | | |
| 16 | Zn₃(PO₄)₂ | 2.1 | 19.3 | 13.9 | 3 | — |
| (comparative) | | | | | | |
| 17 | Zn₃(PO₄)₂ | 2.1 | 24.0 | 14.3 | 3 | — |
| (comparative) | | | | | | |

### Methods

### Test of Breaking Strength

For the measurement of the breaking strength of the shaped bodies, a Zwick Z0.5 tester was used to determine the force needed for breakage. The measurements were carried out in accordance with the standard ASTM D4179. To dry the shaped bodies prior to the measurement, these were stored for at least 3 h at 100° C. in a drying oven and the subsequent measurement of the breaking strength carried out within max. 1 h after the end of drying. The tester was operated in accordance with the standard ASTM D4179 at a constant force rate of 20.0 N/s. For each example, 100 tablets were each positioned individually one after the other with the cylinder axis parallel to the measuring jaw surface (in standard ASTM D4179 the RADIAL CRUSH) and measured. From the 100 individual values, the average value was then determined of the force necessary in each case for breakage of the shaped body, which corresponds to the average breaking force of the shaped body. All data on side crush strength in this application refer to side crush strengths obtained by the method described herein.

### Powder X-Ray Diffraction (XRD)

The catalysts were characterized by X-ray powder diffraction (XRD). In this technique, X-rays undergo diffraction in the crystalline regions of the sample at various diffraction angles. The diffraction angle is measured/plotted in 20. Characteristic reflections occur as a function of the diffraction angle, depending on the phase present. On the basis of these diffraction patterns, an assignment of the diffraction pattern to the phases present can be made with the aid of a database.

The measurements were carried out on a D4 Endeavor from Bruker AXS with Cu-Kα radiation and a LYNXEYE detector. The diffractograms were recorded in a 20 angle range of 5 to 50° with a step width of 0.02° in a recording time of 1.5 s per step with a fixed divergence slit of 0.3°. For the measurement, the samples were finely ground and compressed in a sample holder. The apparatus allowed the diffraction angle to be varied by tilting the sample. All information on XRD reflections in this application refers to XRD reflections obtained with this methodology.

For better comparability of the diffractograms, these were in each case normalized. For this, the data point having the lowest intensity value is first identified in each diffractogram. This value is subtracted from all intensity values of the respective diffractogram. The maximum intensity of the (024) reflection (present at a 20 value of) 28.4-28.5° in the respective diffractogram is then determined. All intensity values of the respective diffractogram are divided by this value.

### Catalytic Test Reaction

To determine catalyst performance, after the preparation of the catalyst was complete (reflux, filtration, vacuum drying, calcining, compacting, tabletting, activation), the catalytic properties of all catalysts were tested in a bench-scale reactor with 1.5 mol % butane in air in a diluted catalyst bed (1:9 mixture of catalyst to inert ceramic rings). The selectivity in respect of maleic anhydride (MA) was determined from the experimental data at a mass GHSV of 5500 l/kg/h.

1-24. (canceled)

25. A VPO catalyst for the oxidation of hydrocarbons with molecular oxygen, the VPO catalyst comprising vanadium, phosphorus and oxygen, wherein the VPO catalyst further comprises ZnO.

26. The VPO catalyst according to claim 25, wherein the VPO catalyst exhibits a powder X-ray diffraction pattern having reflections at 31.7 to 31.9°, 34.3 to 34.5°, and 36.2 to 36.4° in a measurement using a D4 Endeavor from Bruker AXS with Cu-Kα radiation and a LYNXEYE detector, the diffractograms having been recorded in a 2θ angle range of 5° to 50° with a step width of 0.02° in a recording time of 1.5 s per step with a fixed divergence slit of 0.3°.

27. The VPO catalyst according to claim 25, wherein the VPO catalyst has a zinc content in the range of 0.5-7 wt.-%, based on the total weight of the VPO catalyst.

28. The VPO catalyst according to claim 25, wherein the VPO catalyst has a zinc content in the range of 1-5 wt.-%, based on the total weight of the VPO catalyst.

29. The VPO catalyst according to claim 25, wherein the VPO catalyst has a molybdenum content in the range of 0.1-1 wt.-% Mo, based on the total weight of the VPO catalyst.

30. The VPO catalyst according to claim 25, comprising:
0.5% by weight to 7% by weight of Zn,
0% by weight to 0.7% by weight of Mo,
26% by weight to 31% by weight of V,
17% by weight to 21% by weight of P, and
3% by weight to 5% by weight of C,
in each case based on the total weight of the VPO catalyst.

31. The VPO catalyst according to claim 25, wherein the shaped body has a double-alpha shape with a height of 3 mm to 8 mm, a length of 5 mm to 9 mm, a width of 4 mm to 8 mm and an internal hole diameter of 1 mm to 4 mm.

32. The VPO catalyst according to claim 25, wherein the shaped body has a cylindrical shape with a height of 3 mm to 8 mm, an essentially round base surface with a diameter of 3 mm to 8 mm and a central axial opening with a diameter of 1 mm to 3 mm.

33. The VPO catalyst according to claim 25, wherein the shaped body has a side crush strength of more than 25 N, the side crush strength being determined with a Zwick Z0.5 tester using the standard ASTM D4179 at a constant force rate of 20.0 N/s, wherein 100 tablets are each positioned individually and measured with the cylinder axis parallel to the measuring jaw surface so as to determine an average breaking force, which represents the side crush strength.

34. The VPO catalyst according to claim 25, wherein
the VPO catalyst has a zinc content in the range of 0.1-10 wt.-%, based on the total weight of the VPO catalyst; and
the VPO catalyst exhibits a powder X-ray diffraction pattern having reflections at 31.7 to 31.9°, 34.3 to 34.5°, and 36.2 to 36.4° in a measurement using a D4 Endeavor from Bruker AXS with Cu-Kα radiation and a LYNXEYE detector, the diffractograms having been recorded in a 2θ angle range of 5° to 50° with a step width of 0.02° in a recording time of 1.5 s per step with a fixed divergence slit of 0.3°.

35. A process for producing a VPO catalyst according to claim 25, the process comprising:
a) providing a reaction mixture that comprises a V(V) compound, a P(V) compound, optionally a Mo compound, a reductant and a solvent,
b) reducing the V(V) compound with the reductant at least in part to vanadyl hydrogen phosphate so as to obtain a suspension of an intermediate product,
c) filtering the suspension of an intermediate product from step b) so as to obtain an intermediate product,
d) drying and/or calcining the intermediate product at a temperature of max. 300° C. so as to obtain a dried intermediate product, and
e) shaping the dried intermediate product from step d) into a shaped body,
wherein ZnO is added after step c).

36. The process according to claim 35, wherein step d) takes place in two steps d₁) and d₂), wherein during step d₁) the catalyst is dried under reduced pressure at between 90° C. and 140° C. for period between 5 h and 48 h, and during step d₂) it is calcined in nitrogen at between 230° C. and 290° C. for a period of between 5 h and 24 h.

37. The process according to claim 35, wherein the ZnO that is added is a component of a binder having one or more additional constituents.

38. Process for producing a VPO catalyst according to claim 13, characterized in that the binder contains a Mg compound, preferably MgO.

39. The process of claim 35, further comprising, after step e) and after the addition of ZnO, activating the shaped body at a temperature above 200° C., in a gas mixture comprising inert gas, hydrogen and water vapour.

40. A process for producing maleic anhydride by catalytic oxidation of n-butane, the process comprising passing a reactant gas comprising oxygen and n-butane through a reactor containing a bed of VPO catalysts according to claim 25.

41. The process according to claim 40, wherein the bed of the VPO catalysts is present in the reactor at a temperature of between 300° C. and 420° C.

42. The process according to claim 40, further comprising, before passing the reactant gas through the reactor, activating the VPO catalysts at a temperature above 200° C., in a gas mixture comprising inert gas, hydrogen and water vapour,

43. The process according to claim 42, wherein the activation step is carried out at a temperature within a range from 300° C. to 500° C., preferably within a range from 350° C. to 450° C., for a period of from 1 h to 24 h.

44. The process according to claim 43, wherein the activation of the VHP phase is effected in two successive steps e₁) and e₂), wherein during step e₁) the catalyst undergoes activation at a temperature within a range from 250° C. to 350° C. for a period of between 1 h to 8 h and during step e₂) it undergoes activation at a temperature within a range from 350° C. to 450° C. for a period of between 4 h to 20 h.
